(19) United States
(12) Patent Application Publication
(10) Pub. No.: US 2024/0100304 A1
(43) Pub. Date: Mar. 28, 2024

(54) GUIDE EXTENSION CATHETER WITH PERFORATED PERFUSION TUBE AND ALIGNMENT DEVICE FOR SAFE CORONARY STENTING

(71) Applicant: MASSOUD LEESAR, BIRMINGHAM, AL (US)

(72) Inventor: MASSOUD LEESAR, BIRMINGHAM, AL (US)

(21) Appl. No.: 17/936,138

(22) Filed: Sep. 28, 2022

(51) Int. Cl. A61M 25/06

(52) U.S. Cl. CPC A61M 25/0662; A61M 25/01; A61M 2025/0681

(57) ABSTRACT

The present guide extension catheter improves the safety of the coronary stenting procedure and overcomes various safety concerns associated with conventional guide extension catheters, such as stent disruption, compromise in blood flow to the heart (ischemia), and hydraulic dissection of the coronary artery. The guide extension catheter system is insertable into a guide catheter and the coronary artery to provide back-up support during coronary stenting. The guide extension catheter system is configured with a push handle at a proximal section, an alignment device at a middle section, and a perfusion tube positioned distally. The alignment device facilitates in alignment of the stent with the concave-shaped inlet at the proximal end of the perfusion tube for the entrance into the lumen of the perfusion tube. The enhanced surface provided by the concave-shaped inlet facilitates in prevention of the stent disruption during the stenting procedure. The perfusion tube is configured with a plurality of perfusion ports disposed spirally along the entire length of the cylindrically shaped shaft of the perfusion tube to deliver oxygenated blood to the heart during stenting, and to create a directed flow of the blood through the perfusion tube which is beneficial in preventing hydraulic coronary artery dissection and other catastrophic events.

### FIELD OF THE INVENTION

The present invention addresses a novel guide extension catheter system providing an improved coronary stenting safety.

In particular, the present invention is directed to a guide extension catheter configured for insertion into the guide catheter and the heart artery (coronary artery) to provide back-up support for the guide catheter during a coronary stenting procedure and for overcoming safety concerns associated with current guide extension catheters, including stent disruption, compromise in blood flow to the heart (ischemia), and hydraulic dissection of the coronary artery.

More in particular, the subject invention is directed to a guide extension catheter configured with a proximal push handle, an alignment device at a middle section of the guide extension catheter, and a perforated perfusion tube at a distal section of the guide extension catheter, where the alignment device at the middle section operates to align the stent with an inlet at the proximal orifice of the perfusion tube, where the inlet of the perfusion tube is concave—shaped with an enhanced surface area to prevent the stent disruption, where the perfusion tube is perforated with a number of perfusion ports spirally disposed along the perfusion tube's entire shaft to deliver oxygenated blood to the heart during the stenting procedure, and where the perfusion tube operates to prevent the hydraulic dissection of and catastrophic events associated with the coronary artery.

The present invention also is directed to an improved guide extension catheter designed to align the stent with the inlet of the perfusion tube resulting in a successful passage of the stent through the inlet into the perfusion tube and coronary artery, where the perfusion tube in its proximal part has a concave-shaped entry point (the inlet) to provide an easy, catch-free and snag-free entry of the stent into the perfusion tube.

Furthermore, the present invention addresses a guide extension catheter designed to prevent ischemia during the coronary stenting procedure by disposing the perfusion ports along substantially the entire length of the shaft of the perfusion tube to provide a flow of the oxygenated blood to the heart during stenting even with deep advancement into the coronary artery. In this respect, the oxygenated blood in the aorta is pumped to pass through the perfusion ports in the proximal part of the perfusion tube into the inner lumen of the perfusion tube and out from the distal end of perfusion tube, thus providing the oxygenated blood downstream from the aorta to the heart muscle distal to the perfusion tube, and a significant portion of perfusion tube may be advanced into the coronary artery with no risk of distal ischemia.

In addition, the present invention addresses the guide extension catheter designed with a number of perfusion ports disposed along the length of the shaft of the perfusion tube to prevent drop in coronary pressure, and where, after contrast injection, the contrast fluid flows from the proximal perfusion ports to the distal end of the perfusion tube with no risk of the hydraulic dissection of the walls of the coronary blood vessel.

### BACKGROUND OF THE INVENTION

Patients who have blockage (stenosis) of the heart artery (coronary artery disease) may present with chest pain or heart attack. In order to treat the stenosis, coronary intervention is performed by inserting a stent into the coronary artery. The coronary stenting procedure involves inserting a guide catheter through the aorta and into the opening (ostium) of the coronary artery. A guidewire is advanced through the guide catheter and stenosis, the stent is advanced across the stenosis and is deployed to unblock the stenosis. Inserting a stent in patients with complex stenosis can create significant backward force leading to the dislodgement of the guide catheter from the ostium of the coronary artery. This situation can make it difficult or impossible for an interventional cardiologist (surgeon) to treat certain forms of coronary artery disease.

Contemporary stenting involves the treatment of complex stenosis in the coronary arteries. Given that patients are nowadays older and have more comorbidities, prior heart bypass surgery, calcification of the coronary artery, and complete blockage of the coronary artery, stenting of such stenoses requires a high degree of guide catheter support to cross the stenosis. Likewise, the use of the radial artery (artery in the wrist) approach for stenting has now become routine because it is associated with less bleeding as compared to using the leg artery (femoral). Current guide catheters are designed for use via the femoral approach. When these guide catheters are used through the radial artery, they are generally offer less support, and alternative approaches for increasing the guide catheter support are needed.

In order to improve the guide catheter support and deploy the stent, a device called the guide extension catheter has been used with the resulting significant improvement of the stenting success rate. The current guide extension catheters are fabricated by a number of manufacturers and include the GuideLiner catheter (Teleflex), Guidezilla (Boston Scientific Corporation), Telescope (Medtronic), and Guidion (IMDS). In addition, the TrapLiner catheter (Teleflex) is available which has a short (13-cm) rapid exchange catheter portion and a balloon on the push member.

A typical guide extension catheter may be configured as a monorail system. The distal shaft is 25-cm long, which passes over the guidewire in a rapid exchange fashion to provide support for stenting the stenosis. It is 1-french less thick than the guide catheter and is designed to minimize trauma to the coronary artery. The proximal end of the guide extension catheter is attached to a thin stainless steel (the push handle), which exits at the hemostatic valve and is used to push and/or pull the system independent of the guide catheter. The push handle is attached to an alignment device (also referred to herein as an alignment mechanism) in the middle of the guide extension catheter, which is attached to a perfusion tube disposed at a distal shaft. Prior to delivery of a guide extension catheter to a site of interest, a guide catheter and a guidewire are advanced in the artery. The push handle is manipulated by a surgeon to advance the guide extension catheter to the tip of the guide catheter.

The use of guide extension catheters is increasing. Recent studies reported that of 9,525 stenting procedures performed, 3113 (33%) needed a guide extension catheter to successfully perform stenting. Concerns have been raised with respect to the stent disruption or stripping off the balloon catheter during advancing the stent into the current guide extension catheters. If the stent strips off the balloon, the guide extension catheter, balloon, and stent should be removed from the coronary artery. This poses significant risks to a patient, as a sudden occlusion of the coronary artery may occur.

An improvement to the guide extension catheters design is thus needed to provide a successful passage of the stent through the inlet into the perfusion tube and coronary artery.

Despite the improvement of the success of stenting by delivery of the stent deep in the coronary artery allowed by the conventional guide extension catheters, they however may unwantedly interfere with blood flow to the heart and significantly drop coronary pressure. Such an effect on blood flow to the heart is called ischemia. Crossing (jailing) the major side branches of the coronary artery by a guide extension catheter can also reduce blood flow to the side-branch and propagate more ischemia. In addition, as a result of the reduced blow flow, the coronary pressure may drop significantly. Such effects would endanger the patient. In addition, this situation can limit the duration for which the guide extension catheter may be kept in the coronary artery.

FIG. 1 depicts the angiography of the right coronary artery (RCA) 12. The guide catheter 10 is engaged into the right coronary artery (RCA). As shown in FIG. 1, there is a severe stenosis 14 of the bifurcation lesion of distal RCA involving posterior descending artery (PDA) and posterior left ventricular branch (PLV).

FIG. 2 shows the coronary guidewire 18 crossed the stenosis (blockage). An operator tried to advance a stent to the lesion site, but the guide catheter was pulled out of the ostium of the RCA and the stent could not be advanced to the stenosis site (not shown). The operator then advanced a conventional guide extension catheter 16 over the guidewire to the distal RCA to provide enough back-up support for advancing the balloon angioplasty catheter and stent to the stenosis site.

FIG. 3 shows baseline electrocardiograms (ECGs) 20 and 22 before advancing the guide extension catheter, which are normal with no deviation of ST-segments. In addition, FIG. 3 shows baseline mean blood pressure 24, systolic blood pressure 26 was 155 mm Hg, and diastolic blood pressure 28 was 90 mm Hg.

FIG. 4 demonstrates ECGs 30 and 32 after advancing the conventional guide extension catheter into the distal part of RCA. As shown, in the top ECG 30, there is 1 mm ST-segment depression (reciprocal changes). The bottom ECG 32 shows that there is 1 mm ST-segment elevation. These ECGs 30, 32 are indicative of an injury pattern to the heart. In addition, blood pressure significantly dropped to the systolic pressure 34 to 90 mm Hg and the diastolic pressure 36 to 70 mm Hg. The patient developed chest pain and became diaphoretic. Given severe ischemia associated with injury pattern to the heart and a significant drop in the blood pressure, the operator immediately advanced the stent to the lesion site and deployed it (as shown in FIG. 5).

FIG. 5 shows the guide catheter 10, the conventional guide extension catheter 16, and the stent 38 deployment. The guidewire 18 is positioned in the distal part of PDA. As demonstrated in FIG. 4, after advancing the conventional guide extension catheter 16 to the distal part of the RCA (right coronary artery), the patient developed significant ST-segment elevation associated with a significant drop in blood pressure. Given that, the operator immediately advanced the stent, deployed it, and then pulled back the guide extension catheter 16 into the guide catheter 10 to relieve severe ischemia (lack of blood and oxygen) supply to the heart. Unfortunately, all of the current FDA approved guide extension catheters induce significant drop in coronary pressure, ST-segment elevation, and ischemia. The consequences of these untoward events could be serious as follows: (a) if the operator cannot advance the stent to the lesion site, the guide extension catheter would need to be retracted immediately to relief ischemia and hypotension, (b) given the operator needs to advance stent urgently to the lesion site and deploy it, there would not be enough time to accurately position the stent at the lesion site, (c) given the urgent deployment of the stent, some part of blockage may not be covered (geographic miss). As a result of geographic miss, covering the entire lesion with a second stent will be even more challenging because of severe hemodynamic compromise after re-advancing the guide extension catheter and passing the second stent through the first stent, which could be potentially challenging, and (d) if the second stent could not be successfully positioned distal to the first stent, the patient may develop severe ischemia and hemodynamic compromise. This might lead to immediate coronary artery bypass surgery if the patient survives to operating room and surgery.

While the guide extension catheter has significantly improved the success of stenting, they can pose a significant risk associated with ischemia, cardiogenic shock, heart attack, severe rhythm problem, and death. In addition, since stent needs to be advanced and deployed quickly, it might increase complications. This kind of procedure requires a very high degree of expertise, and the operator should be extremely quick in deploying the stent. In this respect, a safer guide extension catheter is urgently needed to maintain oxygen supply to the heart while providing back-up support for complex coronary interventions. Any delay in advancing and deploying the stent may propagate the extent of ischemia, which may lead to heart attack, ventricular fibrillation, and death.

FIG. 6 shows that the stent was positioned in the RCA with final kissing balloon inflation 14. The guide extension catheter was removed from the RCA, and the procedure was completed successfully. In this case distal RCA was not calcified and the stent was advanced with no resistance. In situation where there is significant amount of calcium at the lesion site, deployment of the stent in the distal lesion can be extremely challenging.

The major limitations of the current guide extension catheters are that they may interfere with blood flow to the heart (ischemia) and drop in coronary pressure. After insertion of the guide extension catheter into the coronary artery, if the stent does not advance to the lesion site, the guide extension catheter and stent will need to be pulled back to guide catheter to reduce the extent of ischemia. Failure to deploy the stent could lead to sudden coronary occlusion with associated cardiogenic shock and heart attack. Associated with the myocardial ischemia, the balloon inflation in the coronary artery may lead to ST-segment elevation, chest pain, wall motion abnormalities assessed by echocardiography, reduction in ejection fraction, and a rise in lactate level in the coronary sinus even with 2 minutes of ischemia. Prolongation of the ischemia could lead to serious consequences including heart attack, ventricular fibrillation, and death.

A study showed that that the auto perfusion balloon angioplasty catheter (“The Auto perfusion balloon” disclosed in U.S. Pat. No. 5,087,247) limited myocardial ischemia and necrosis in dogs. This balloon is no longer available, but the presence of side holes formed proximally and distally to the balloon shaft, resulted in the antegrade blood flow from the proximal to distal part of the balloon catheter. This significantly improved regional myocardial blood flow compared with the standard balloon angioplasty catheter. Likewise, the degree of the ST-segment elevation was significantly lower with the auto perfusion balloon vs. the standard balloon angioplasty catheter during 3 minutes of balloon inflation. The side holes located in the proximal and distal parts of the balloon significantly reduced the extent of ischemia during the balloon angioplasty. In addition, a study with the auto perfusion balloon catheter showed that it reduced the chest pain and ECG changes during balloon angioplasty in humans.

Another perfusion balloon angioplasty catheter (U.S. Pat. No. 7,087,039) replicated the same results. The auto perfusion balloons reduced ischemia because perfusion ports disposed in the proximal and distal shaft of the balloons support the oxygenated blood delivery to the heart.

It is therefore desirable to prevent ischemia situation in the stenting procedure by improving a guide extension catheter with perfusion ports disposed along the entire shaft of the perfusion tube.

As presented supra, there are currently 4 kinds of guide extension catheters on the market. The design of all present guide extension catheters is virtually the same, except for the TrapLiner, which has shorter guide extension (13 cm vs. 25 cm for others). The usage of the conventional guide extension catheters significantly drops coronary pressure as well as the ischemia and chest pain during the coronary stenting. They require skilled operators to quickly advance these catheters.

O'Connell et al. (U.S. Patent Application Publication No. 2017/0080178) discloses a guide extension catheter in which openings are fabricated in the distal part of the distal shaft (4-6 cm). Such openings are expected to result in fluid communication between an area outside the guide extension catheter and the lumen and prevent a drop in coronary pressure. However, there are some limitations of the O'Connell design, including: (a) if the catheter is embedded in a tight blockage in the coronary artery, there will not be space between the catheter and an area outside the catheter for fluid communication, (b) given there are no perfusion ports in the proximal part of the distal shaft, there will not be antegrade perfusion from the proximal to the distal shaft and the heart muscle at the end of catheter, and (c) only 4-6 cm of the distal shaft are fenestrated, while the guide extension catheter is frequently pushed further down into the coronary artery, and the oxygenated blood cannot be delivered to the heart if the catheter is inserted deeper than 6 cm into the coronary artery.

In this respect, an improvement to O'Connell is desirable in which the perfusion tube would be fabricated with perfusion ports spread along its entire shaft to provide oxygenated blood to the heart during stenting even with deep advancement into the coronary artery, so that the oxygenated blood in the aorta is pumped to pass through the perfusion ports in the proximal part of the perfusion tube into the inner lumen of the perfusion tube and out from the distal end of perfusion tube, thus providing the oxygenated blood downstream from the aorta to the heart muscle distal to the perfusion tube. In addition, as a further improvement to the O'Connell's system, it would be desirable that the operator advances a significant portion of perfusion tube into the coronary artery with no risk of distal ischemia.

After advancing the current guide extension catheters into the coronary artery, coronary pressure significantly drops and dampening of pressure occurs (as presented in FIG. 4). In this context, injection of contrast media through the guide catheter may induce and propagate hydraulic dissection in the coronary artery, which results in coronary occlusion or perforation. Coronary occlusion or perforation is devastating and is associated with a high rate of mortality.

It is therefore desirable to fabricate the perfusion ports along the entire shaft of the perfusion tube to prevent a drop in coronary pressure. After the contrast fluid injection, the contrast fluid flows from proximal perfusion ports to the distal end of perfusion tube with no risk of hydraulic dissection.

Overall, there is a long-lasting need for highly safe cardiac stenting procedure by providing an improved guide extension catheter designed for permitting the perfusion of the oxygenated blood to the heart and for preventing ischemia and a drop in coronary pressure.

### SUMMARY OF THE INVENTION

It is therefore an object of the present invention to provide a guide extension catheter system allowing an improved coronary stenting safety.

It is another object of the present invention to provide a guide extension catheter configured for insertion into the guide catheter and the heart artery (coronary artery) to provide back-up support for the guide catheter during a coronary stenting procedure and for overcoming safety concerns associated with current guide extension catheters, including stent disruption, compromise in blood flow to the heart (ischemia), and hydraulic dissection of the coronary artery.

It is a further object of the present invention to provide a guide extension catheter configured with a proximal push handle, an alignment device at a middle section of the guide extension catheter, and a perforated perfusion tube at a distal section of the guide extension catheter, where the alignment device at the middle section operates to align the stent with an inlet at the orifice of the perfusion tube, where the inlet of the perfusion tube is concave—shaped with an enhanced surface area to prevent the stent disruption, where the perfusion tube is perforated with a number of perfusion ports spirally disposed along the perfusion tube's entire shaft to deliver oxygenated blood to the heart during the stenting procedure, and where the perfusion tube operates to prevent the hydraulic dissection of and catastrophic events associated with the coronary artery.

In addition, it is an object of the present invention to provide an improved guide extension catheter designed to align the stent with the inlet of the perfusion tube resulting in a successful passage of the stent through the inlet into the perfusion tube and coronary artery, where the perfusion tube in its proximal part has a concave-shaped entry point (the inlet) to provide an easy, catch-free and snag-free entry of the stent into the perfusion tube.

Furthermore, it is an object of the present invention to provide a guide extension catheter designed to prevent ischemia during the coronary stenting procedure by disposing the perfusion ports along substantially the entire length of the shaft of the perfusion tube to provide a flow of the oxygenated blood to the heart during stenting even with deep advancement into the coronary artery. In this respect, the oxygenated blood in the aorta is pumped to pass through the perfusion ports in the proximal part of the perfusion tube into the inner lumen of the perfusion tube and out from the distal end of perfusion tube, thus providing the oxygenated blood downstream from the aorta to the heart muscle distal to the perfusion tube, and a significant portion of perfusion tube may be advanced into the coronary artery with no risk of distal ischemia.

It is an additional object of the present invention to provide a guide extension catheter having the perfusion ports positioned along the entire shaft of the perfusion tube to prevent a drop in coronary pressure, and were, after contrast injection, the contrast flows from the proximal perfusion ports to the distal end of the perfusion tube with no risk of hydraulic dissection.

The present invention addresses a novel guide extension catheter system which is composed of (a) an elongated tubular member (push handle) positioned in the proximal segment of the guide extension catheter, (b) a perfusion tube at the distal section of the guide extension catheter, and (c) an alignment device (mechanism) at the middle section connected to the inlet at the beginning of the perfusion tube, where the push handle has a cross-sectional outer diameter smaller than the cross-sectional inner diameter of the guide catheter. The proximal portion of the push handle extends proximally through a hemostatic valve, which is attached to the guide catheter.

The alignment device in the present guide extension catheter is connected to the push handle proximally and to the perfusion tube distally. The alignment device facilitates a coaxial longitudinal advancement of the stent into the inlet of the perfusion tube. The inlet of the perfusion tube is concave shaped with the purpose of significantly increasing the surface area of the perfusion tube's proximal inlet and to prevent the stripping of the stent and the snagging of an interventional device to the wall of the perfusion tube's inlet.

The present guide extension catheter enhances the perfusion of the heart during coronary stenting procedure. The perfusion tube in the subject guide extension catheter is configured with several perfusion ports disposed spirally along the entire length of the perfusion tube's shaft to maintain the coronary blood flow and to deliver the oxygenated blood to the heart muscle during the coronary stenting procedure. One of the important features of the present guide extension catheter is that the perfusion tube is able to provide oxygenated blood to the heart muscle even with a deep intubation of the guide extension catheter into the coronary artery.

Another uniqueness of the present guide extension catheter relates to the prevention of hydraulic dissection of the coronary artery. After the insertion of the current guide extension catheters into the coronary artery, the coronary pressure drops. In this setting, a contrast injection is typically performed through the guide catheter which may lead to the hydraulic dissection of the coronary artery when a conventional guide extension catheter is used. However, in the present system, with the perfusion ports disposed along the entire shaft of the perfusion tube, a drop in coronary pressure or pressure dampening is prevented. After the contrast injection, the contrast fluid flows from the proximal perfusion ports to the distal end of the subject perfusion tube with no risk of hydraulic dissection.

In one aspect, the present invention concerns a guide extension catheter system advanceable through a guide catheter to provide back-up support for coronary stent deployment in a blood vessel. The subject system includes (a) a push handle disposed at the proximal section, (b) an alignment device disposed at the in the middle section, and (c) a perfusion tube disposed at the distal section.

The perfusion tube preferably has a cylindrically shaped shaft extending between a proximal end and a distal end. The cylindrically shaped shaft has an internal lumen and a wall defining the internal lumen. Of importance, the proximal end of the cylindrically shaped shaft of the perfusion tube is configured with a concave-shaped inlet. It is also of importance to the purposes and objectives of the present system that the perfusion tube is configured with a plurality of perfusion ports formed in the wall of the cylindrically shaped shaft. The plurality of perfusion ports is preferably disposed spirally around and along an entire length of the cylindrically shaped shaft of the perfusion tube.

For the coronary stenting, a stent system is insertable longitudinally in the lumen of the cylindrically shaped shaft and is displaceable therealong between the proximal section of the guide extension catheter system and beyond the distal end of the cylindrically shaped shaft of the perfusion tube. The guide catheter is also used which is configured to receive the guide extension catheter longitudinally therein for displacement therealong. For the coronary procedure, the stent enters the lumen of the perfusion tube through the concave—shaped inlet formed at the proximal end of the cylindrically shaped shaft of the perfusion tube. The perfusion tube has a cross-sectional outer diameter smaller than a cross-sectional outer diameter of the guide catheter.

The perfusion ports formed in the wall of the cylindrically shaped shaft of the perfusion tube provide a directed fluid communication from the proximal end to and through the distal end of the perfusion tube. When the perfusion tube is insertable into the coronary artery, the plurality of perfusion ports support delivery of oxygenated blood to the heart muscle.

In the present system, the push handle has an outer diameter smaller than an outer diameter of the perfusion tube, and a length of the push handle combined with the lengths of the cylindrically shaped shaft of the perfusion tube and a length of the alignment device forms a collective length of the guide extension catheter which exceeds a length of the guide catheter.

The push handle is configured to transfer an action applied thereto to the alignment device and to the perfusion tube to advance the perfusion tube into the coronary artery. When the guide extension catheter is disposed in the guide catheter, the cylindrically shaped shaft of the perfusion tube extends out of a distal end of the guide catheter, and a proximal end of the push handle extends out of the guide extension catheter at its proximal section.

It is preferred that the cylindrically shaped shaft of the perfusion tube is configured with an outer layer and inner layer formed in contact with an outer surface and an inner surface, respectively, of the wall of the cylindrically shaped shaft. The outer layer has a hydrophilic coating for smooth passage of the perfusion tube into the blood vessel.

The cylindrically shaped shaft of the perfusion tube further comprises a reinforcement structure configured with spiral coils embedded between the outer and inner layers of the cylindrically shaped shaft of the perfusion tube to prevent kinking or collapse of the perfusion tube during advancing into the blood vessel. The perfusion ports are preferably disposed between adjacent windings of the spiral coils of the cylindrically shaped shaft of the perfusion tube. The perfusion ports may be configured in a variety of geometric shapes, including a round shape, and may have a diameter ranging from 0.014 to 0.020 inches. The perfusion ports are disposed along the cylindrically shaped shaft of the perfusion tube to maintain coronary perfusion and to prevent hydraulic dissection of the coronary artery and heart attack.

The cylindrically shaped shaft of the perfusion tube further includes a reinforcing polymer layer secured to the wall of the cylindrically shaped shaft at its proximal end and having a length of approximately 2 cm. The reinforcing polymer layer provides strength, pushability and flexibility to the perfusion tube to support passage of the stent into the perfusion tube.

The cylindrically shaped shaft of the perfusion tube is formed with a distal tip (approximately 2 mm in length) at the distal end of the cylindrically shaped shaft. The distal tip is preferably formed from a soft radiopaque material to provide atraumatic advancement of the perfusion tube into the blood vessel.

A diameter of the lumen of the cylindrically shaped shaft of the perfusion tube is sufficient for passage of interventional devices therethrough and for supporting sufficient perfusion to the heart.

The alignment device comprises a U-shaped polymer member and the concave-shaped inlet in direct coupling with the U-shaped polymer member. The distal end of the U-shaped polymer member is incorporated in the concave-shaped inlet. A top portion of the U-shaped polymer member is cutout to prevent wrapping of the guidewire and to facilitate the passage of interventional devices to the concave-shaped inlet without entanglement.

The concave-shaped inlet at the proximal end of the perfusion tube is preferably formed with a polymer material to maintain flexibility of and to prevent collapse of the of lumen of the cylindrically shaped shaft.

In another aspect, the present invention addresses a method for intravascular treatment of a coronary artery blockage, which comprises the following steps:
(a) advancing a guide catheter through aorta into the ostium of the coronary artery;
(b) advancing a guidewire through a lumen of the guide catheter into the coronary artery and crossing beyond the blockage;
(c) assembling a guide extension catheter having a push handle disposed at a proximal section of the guide extension catheter, an alignment device at a middle section of the guide extension catheter, and a perfusion tube at a distal section of the guide extension catheter, the perfusion tube having a concave-shaped inlet at a proximal end thereof and a plurality of perfusion ports disposed spirally along a length of the perfusion tube;
(d) advancing the push handle to position the perfusion tube beyond a distal end of the guide catheter into the coronary artery by using the guidewire as a rail;
(e) advancing a balloon angioplasty catheter over the guidewire through the alignment mechanism, and positioning the perfusion tube proximal to the blockage in the coronary artery; and
(f) advancing a stent over the guidewire through the alignment mechanism and the perfusion tube into the coronary artery and positioning the stent across the blockage to unblock the blockage.

The subject method further comprises the step of:
in the step (e), advancing a guidewire and interventional devices through the alignment mechanism into the perfusion tube with no snagging or stripping off of the stent from balloon angioplasty catheter;
in the step (d), advancing the perfusion tube into the coronary artery distal to the side-branch and positioning it proximal to the stenosis; and
in the step (e), configuring blood flow from the heart and passing through the aorta, proximal coronary artery, perfusion ports, perfusion tube, distal coronary artery, coronary microvasculature and heart tissue.

In the present method, continuous perfusion of the distal coronary artery is maintained by functionality of the perfusion tube to reduce the risk of ischemia of the heart muscle.

In addition, the present method provides for maintaining perfusion from a proximal end to a distal end of the perfusion tube, thus preventing blood pressure drift from a hub of the guide catheter to a distal tip of the perfusion tube. It is of importance, that the subject method attains supply of the oxygenated blood to a side-branch by the perfusion ports to preventing ischemia in the side-branch, as well as maintains continuous perfusion of the distal coronary artery by the perfusion tube, thus reducing the risk of hydraulic dissection with contrast injection.

These and other objects and advantages of the present disclosure will become more apparent in view of the patent Drawings and the following description of the preferred embodiment(s).

### BRIEF DESCRIPTION OF THE DRAWINGS

FIG. 1 demonstrates a typical angiography procedure of the right coronary artery in a patient with a critical stenosis of posterior descending artery as shown by arrow;

FIG. 2 depicts a conventional guide extension catheter advanced to the distal part of right coronary artery to support advancing the stent;

FIG. 3 is a diagram representative of a baseline ECG and blood pressure before advancing the conventional guide extension catheter to the distal part of the right coronary artery;

FIG. 4 demonstrates a ST-segment elevation in the ECG and BP drop following the advancing of the conventional guide extension catheter into the distal part of the right coronary artery:

FIG. 5 depicts the procedure after advancing of the conventional guide extension catheter to the distal part of the right coronary artery, and a stent immediately deployed to the PDA;

FIG. 6 illustrates coronary stenting with the stent successfully deployed and the conventional guide extension catheter pulled back into the guide catheter;

FIG. 7 is a schematic representation of the subject guide extension catheter;

FIG. 8 depicts a schematic representation of the subject guide extension catheter inserted into the guide catheter, where the stent is shown when passed the orifice (inlet) of the perfusion tube;

FIG. 9 shows a cross section taken along the lines 4-4 of FIG. 8;

FIG. 10 shows a cross section taken along the lines 5-5 of FIG. 8;

FIG. 11 depicts a spiral coil encapsulated within the perfusion tube;

FIG. 12 shows an inner layer of the perfusion tube, which is inserted inside the spiral coil;

FIG. 13 shows the perfusion tube with the spiral coils, perfusion ports, inner and outer layers, and the inlet;

FIG. 14 depicts schematically a partial cut-away of the spiral coil and the perfusion tube;

FIG. 15 depicts schematically the perfusion tube positioned inside the guide catheter and advanced into the heart artery. As the result of perfusion through the perfusion ports in the perfusion tube, blood pressure measured at the hub of guide catheter is not dampened; and

FIG. 16 schematically shows directions of blood flow from the heart to the aorta, perfusion ports, perfusion tube, side-branch, and heart tissue.

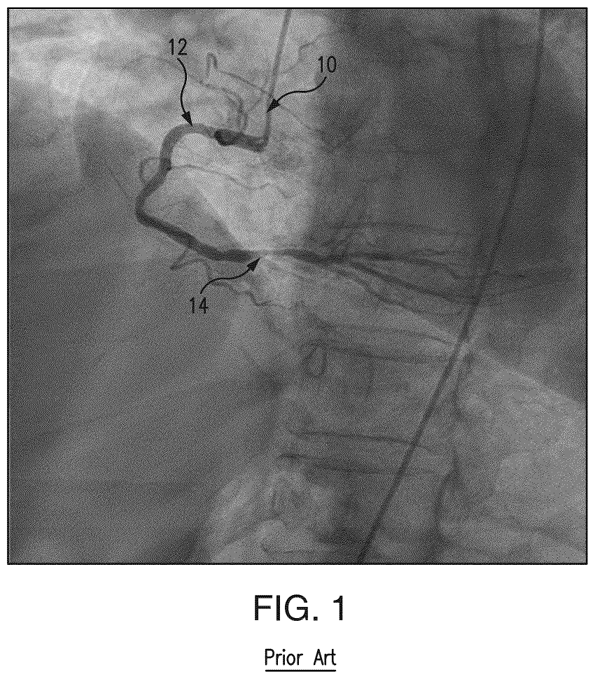

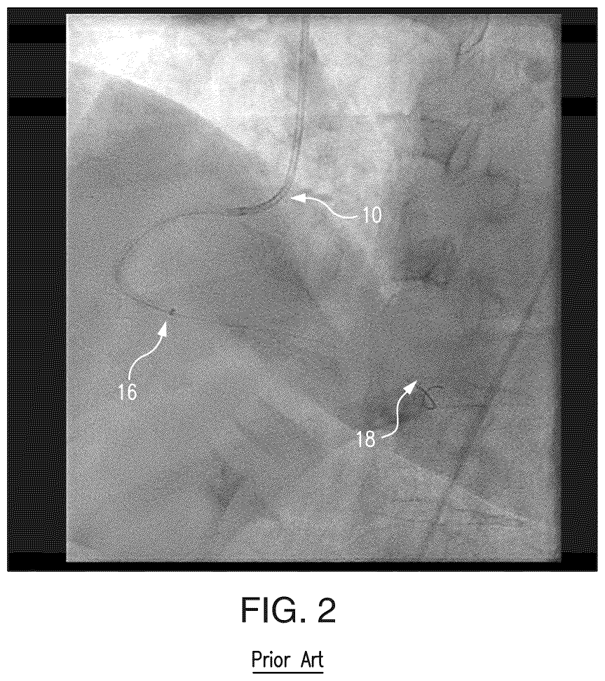

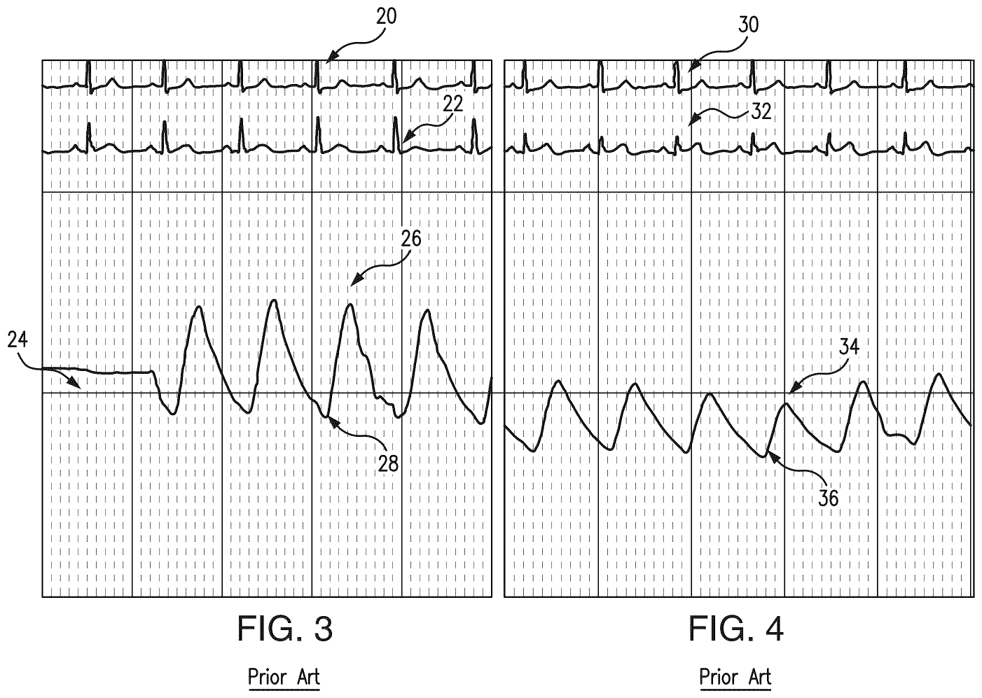

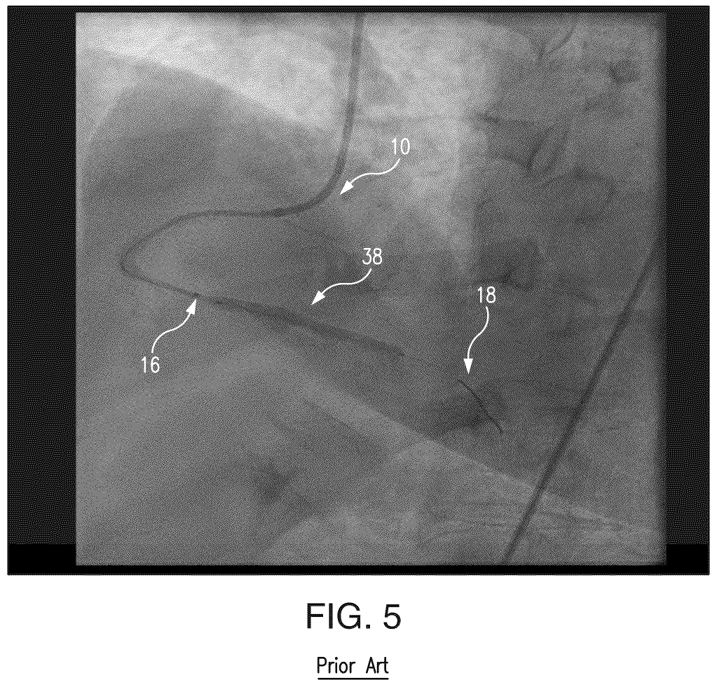

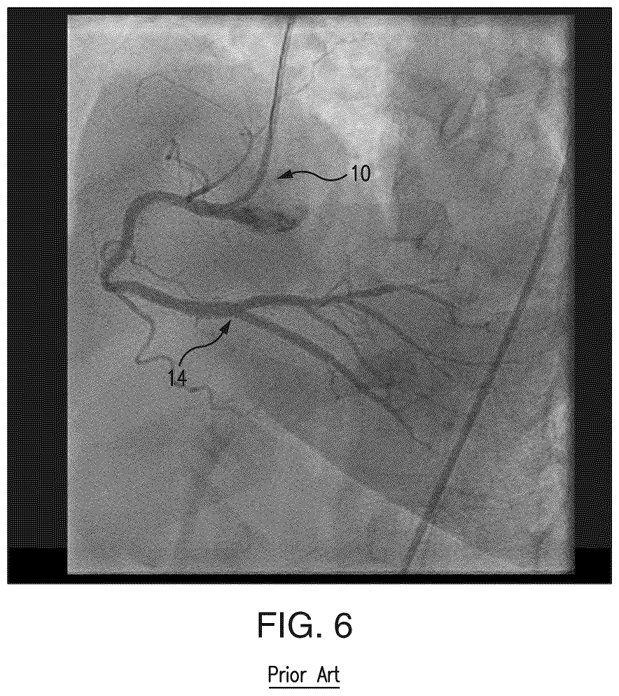

### DETAILED DESCRIPTION OF THE PREFERRED EMBODIMENT(S)

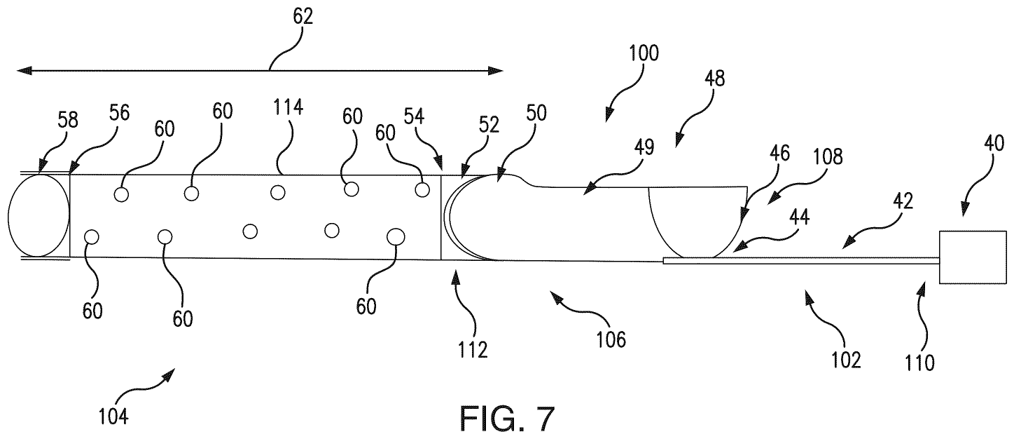

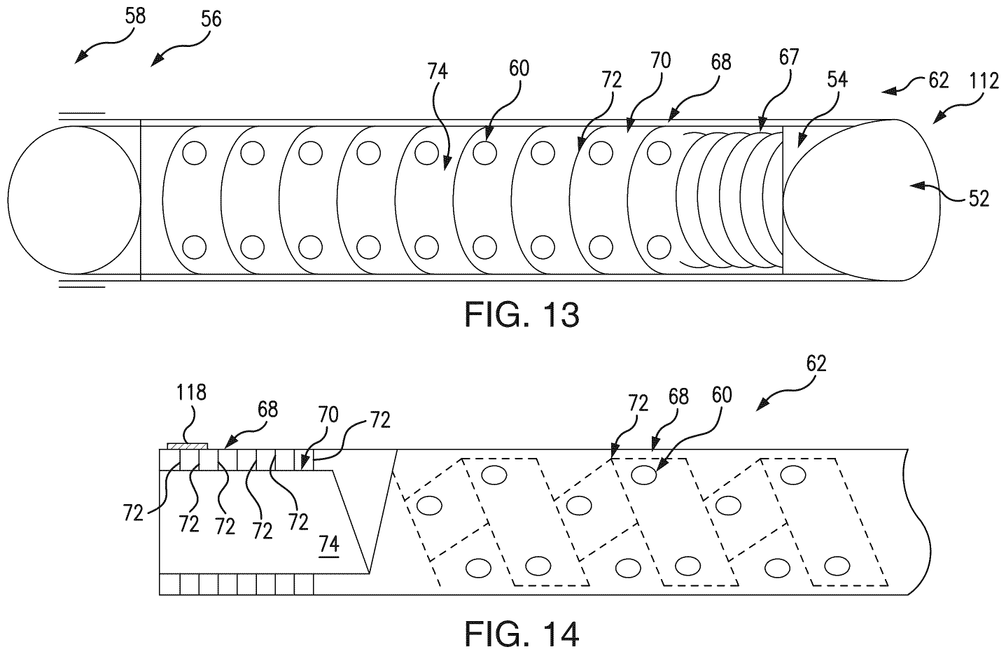

FIGS. 7-16 depict the illustrative aspect of embodiments and are not intended to limit the scope of invention. The detailed description of drawings is not scaled, and similar elements are numbered the same in different illustrations. The term “proximal” means any equipment closer to the body and the term “distal” means the equipment of discussion is further away from the body or heart or aorta.

FIGS. 7-10 represent schematic views of the subject guide extension catheter system 100 which has a proximal section 102, a distal section 104, and a middle section 106 disposed between the proximal section 102 and the distal portion 104 of the current guide extension catheter 100. The guide extension catheter system 100 includes a push handle 42 at the proximal section 102 of the guide extension catheter 100, an alignment device 48 positioned at the middle section 106, and a perfusion tube 62 disposed at the distal section 104.distally.

A distal portion 44 of the push handle 42 is connected to the wall 46 at the proximal end 108 of the alignment device 48. A distal end 50 of the alignment device 48 is connected 50 to the perfusion tube 62.

The push handle 42 may be fabricated from a metal, or Nitinol, and has a knob 40 at the proximal end 110, which remains outside of the guide catheter 10. The push handle 42 is configured to transfer a push and/or pull manipulation applied by a surgeon (operator) through the knob 40 to the alignment device 48, and ultimately, to the perfusion tube 62 to advance the perfusion tube 62 into the coronary artery.

The perfusion tube 62 has a proximal orifice and a distal orifice at the proximal end 112 and the distal end 58, respectively, of the perfusion tube 62. The proximal end 112 of the perfusion tube 62 is configured with a concaved shaped inlet 52 which cooperates with the proximal orifice. Shown in FIGS. 7, 8, 11, and 13, marker bands 54 and 56 are positioned in proximity to the proximal end 112 and the distal end 58, respectively, of the perfusion tube 62 to determine the position of the perfusion tube 62 by fluoroscopy after advancing the perfusion tube 62 into the guide catheter 10. The marker bands 54 and 56 are made of platinum or other radiopaque material and are attached to the perfusion tube 62 by crimping. The proximal marker band 54 is disposed at the inlet 52, and the distal marker 56 is disposed just before the soft 2 mm long distal tip 58. The distal tip 58 of the perfusion tube 62 may have a length, approximately, of 2 mm and is fabricated from a soft plastic material, which provides the atraumatic advancement of the perfusion tube into the coronary artery to reduce the risk of dissection when in contact with the arterial wall.

As shown in FIGS. 8, the subject guide extension catheter 100 is inserted into the guide catheter 10 through the hemostatic device 64. The guide extension catheter 100 may have the length of 150 cm, while the guide catheter 10 may have a length between 90 and 100 cm, so that the distal section 104 of the guide extension catheter 100 extends outside of the guide catheter 10. The diameter of guide extension catheter 100 changes along its length and is larger at the perfusion tube 62 than at the push handle 42. The outer diameter of the guide extension catheter 100 is smaller than the inner diameter of the guide catheter 10, so that the guide extension catheter 100 is insertable through the guide catheter 10. For example, the inner diameter of the 6 French (F) guide catheter 10 may be about 0.070-0.071″ (1F=⅓ mm), while the outer diameter of a 6F guide extension catheter 100 may be 0.067″, so that there is 0.03-0.04″ difference in diameters between the inner diameter of the guide catheter 10 and the outer diameter of guide extension catheter 100 to coaxially insert the guide extension catheter inside the guide catheter.

As shown in FIGS. 7, 8 and 9, the alignment device 48 is composed of a U-shaped polymer member 49 and a concaved shaped inlet 52 of the perfusion tube 62. The distal end 50 of the U-shaped polymer member 49 is coupled (glued, adhered, or welded) to the inlet 52.

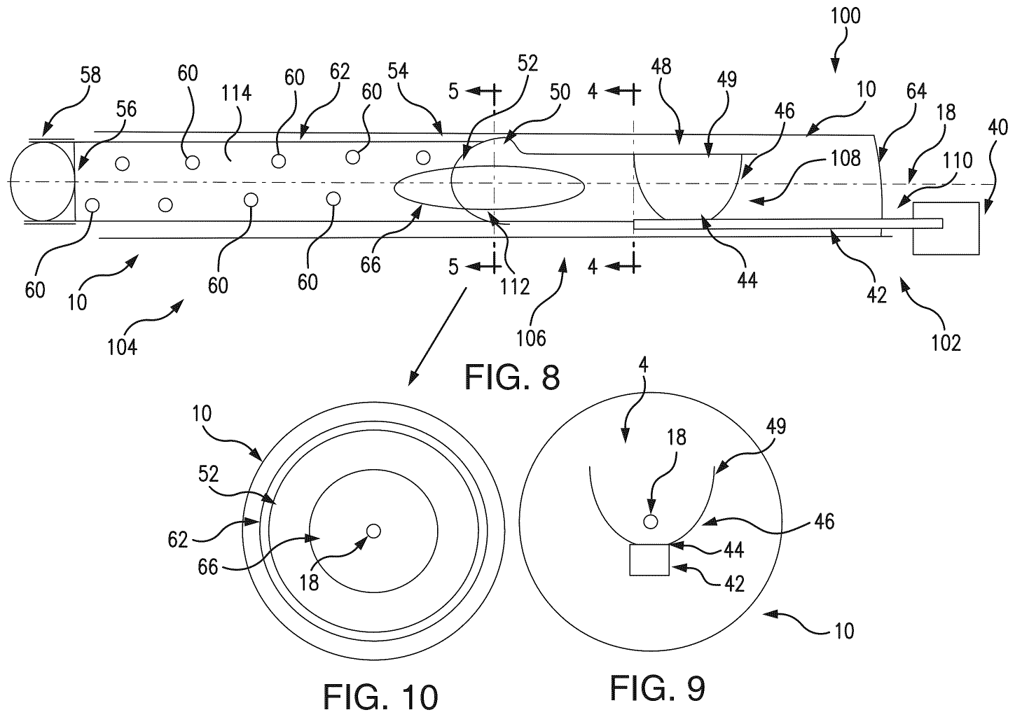

FIG. 9 shows a cross-sectional view of the U-shaped polymer member 49 taken along the lines 4-4 of FIG. 8. The U-shaped polymer member 49 provides a direct path to the inlet 52. The top part of the U-shaped polymer member 49 is cutout to prevent guidewire wrapping and to facilitate the passage of interventional devices to the inlet 52 without entanglement. The guide extension catheter 100 has a unique design in several aspects. For example, one of the novel features includes the U-shaped polymer member 49 which provides more depth than the half-pipe design used in the conventional guide extension catheters to prevent the entanglement of interventional devices. Along the same line, the design of the concave-shaped polymer inlet 52 is novel in that it provides an expanded surface area which is larger than the polymer collar used in the conventional guide extension catheters to prevent stent distortion or stripping.

The subject guide extension catheter 100 overcomes the shortcomings of the conventional guide extension catheters with respect to stent distortion or stent stripping off of the balloon angioplasty catheter which require removal from the body of the entire system including the guide extension catheter and the stent. The present guide extension catheter 100 advantageously introduces the alignment device 48 (the U-shaped polymer member 49 and the inlet 52). The U-shaped polymer member 49 is designed to reduce the risk of wire wrapping or entangling of interventional devices. The concaved shaped inlet 52 has a surface area larger than that of the current guide extension catheters to reduce the risk of stent snagging to the wall with subsequent stent distortion or stripping. In this respect, the present invention is an improvement over current guide extension catheters in that the current system reduces the risk of interventional device entanglement, as well as stent disruption and stripping.

The distal end 44 of the push handle 42, as shown in FIGS. 7, 8 and 9, may be welded (glued, adhered) to the wall 46 at the proximal end 108 of the of the alignment device 48. As shown in FIGS. 8, 9 and 10, the guidewire 18 is advanced through the hemostatic valve 64 alongside the push handle 42 inside the guide catheter 10. The guidewire 18 is passed through the U-shaped polymer member 49 to the inlet 52 and the coronary artery. Likewise, the stent 66 is passed through the U-shaped polymer member 49 to the inlet 52, perfusion tube 62, and the coronary artery. FIG. 10 is a cross-sectional view taken along lines 5-5 of FIG. 8. As shown in FIG. 10, the alignment device 48 directs (centers) the guidewire 18 and stent 66 into the inlet 52 of the perfusion tube 62.

In an embodiment shown in FIG. 8, the inlet 52 has a concaved shaped entry. This would significantly increase the surface area of the inlet 52 for receiving the stent 66 thereinto. In addition, the inlet 52 is constructed with polymer (plastic) rather than metal. These two modifications, as compared with the conventional guide extension catheters, would prevent a destructive interaction between stent/inlet, which otherwise could lead to stent disruption or stripping.

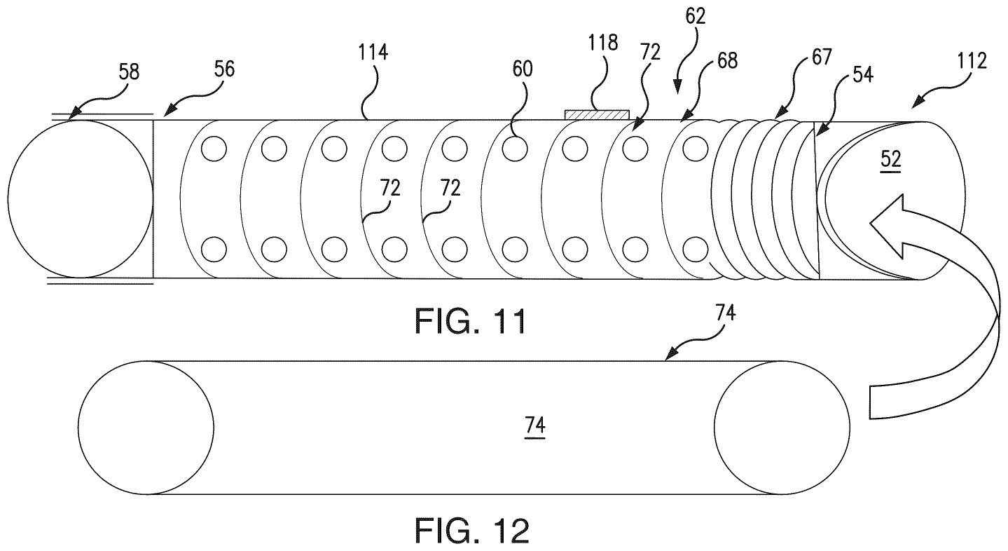

FIGS. 11-14 detail the novel perfusion tube 62 of the present guide extension catheter 100. As shown in FIG. 11, the 2 cm long proximal part at the proximal end 112 of the perfusion tube 62 is constructed from a reinforced polymer member 67 to provide a sufficient strength in combination with flexibility which would be beneficial for the passage of the stent 66 into the perfusion tube 62. FIG. 12 displays the inner layer 70 of the perfusion tube 62. The inner layer 70 is tube shaped and is fabricated from a material having flexibility. The material for the inner layer 70 may be selected from urethane or polyamide elastomer to prevent friction during advancing of interventional devices. The perfusion tube 62 also has an outer layer 68 which may be configured from materials such as nylon.

A support system of the perfusion tube 62 is composed of a thin wire spiral coil 72 (with a wire thickness of approximately 1 mil to 3 mils) made from a shape memory alloy, such as, for example, Nitinol, to provide strength and to prevent kinking of the tubular shaft of the catheter 100. The spiral coil 72 is embedded in the jacket between the inner layer 70 and the outer layer 68. The inner and outer layers 70, 68 are coated with a thin layer of a plastic material. The outer layer 68 has hydrophilic coating 118 for the smooth passage of guide extension catheter 100 in the coronary artery. This would enable the perfusion tube to be smooth such that it can be advanced through tortuous and calcified arteries. The spiral coil 72 is secured between the outer and inner layers 68, 70 by means of heat bonding or the fusion technique. This provides a sufficient support to prevent kinking or lumen collapse. The lumen 74 of the perfusion tube 62 has an inner diameter sufficient to deliver therethrough various devices and provide perfusion to the heart.

The perfusion tube 62 is configured with perfusion ports 60 along its entire length passed the reinforced polymer in the proximal part 112. The perfusion ports 60 are extended from the outer layer 68 to inner layer 70 of the perfusion tube 62. The perfusion ports 60 may be fabricated by laser cutting or mechanical drilling. As depicted in FIG. 11, the perfusion ports 60 are disposed between the windings of the spiral coils 72 of the perfusion tube 62. Perfusion ports 60 serve to transfer blood from the aorta and coronary artery into the lumen 74 of the perfusion tube 62. In addition, the perfusion ports 60 transfer oxygenated blood to the low-pressure regions around the perfusion tube 62 and to the branches of the coronary artery covered by the perfusion tube.

The perfusion ports 60 may be configured in a variety of geometric shapes, for example, having a round contour. The size of the perfusion ports 60 may be, for example, between 0.014 and 0.020 inches in diameter. Similar to the direction of the spiral coil 72, the perfusion ports 60 may be arranged spirally along the axes of the spiral coils. This may improve the flexibility of the perfusion tube 62.

An additional benefit of the spiral locations of the perfusion ports 60 is prevention of the issues encountered with blocking of perfusion to the heart. If all perfusion ports were disposed at one side of the shaft 114 of the perfusion tube 62, the perfusion would be blocked after leaning the perfusion tube 62 against and in contact with the wall of the artery. Spiral disposition of the perfusion ports 60, as designed in the present guide extension catheter 100, ensures a sufficient number of unblocked (unobstructed) ports 60 always available, and the perfusion flow maintained therethrough.

The length of the perfusion tube 62 may be, for example, approximately 14 cm. The length of the perfusion tube to be 14 cm is an example only which may provide an effective perfusion to the heart. However, different dimensions of the perfusion tube 62 are also contemplated in the present system 100, and longer tubes 62 may be needed for bypass grafts.

The advantage of having perfusion ports 60 along the entire length of the perfusion tube 62 is that the surgeon (operator) is not limited by advancing of the perfusion tube only a few centimeters to the artery. In this respect, the present design ensures to preserve perfusion along the length of the perfusion tube.

An intravascular ultrasound has been performed to measure the length of the coronary artery and determined that the average length of the coronary artery is between 12-14 cm. The average length of 6 to 10 cm of the perfusion tube 62 was estimated to be needed to provide perfusion to the heart during stenting for lesions disposed between the middle to the distal part of the coronary artery, respectively. The perfusion of the oxygenated blood deep into the coronary artery provided by the present system, is not available by conventional guide extension catheters.

The perfusion tube 62, as illustrated in FIG. 14, is configured with the spiral coils inside 72, which are encapsulated within the flexible inner layer 70 and the outer layer 68. The interior of lumen 74 provides blood passage inside the perfusion tube 62. The spiral coils 72 support the lumen 74 against collapse. Perfusion ports 60 are spirally disposed between the helical coils 72 along the entire length of the perfusion tube 62.

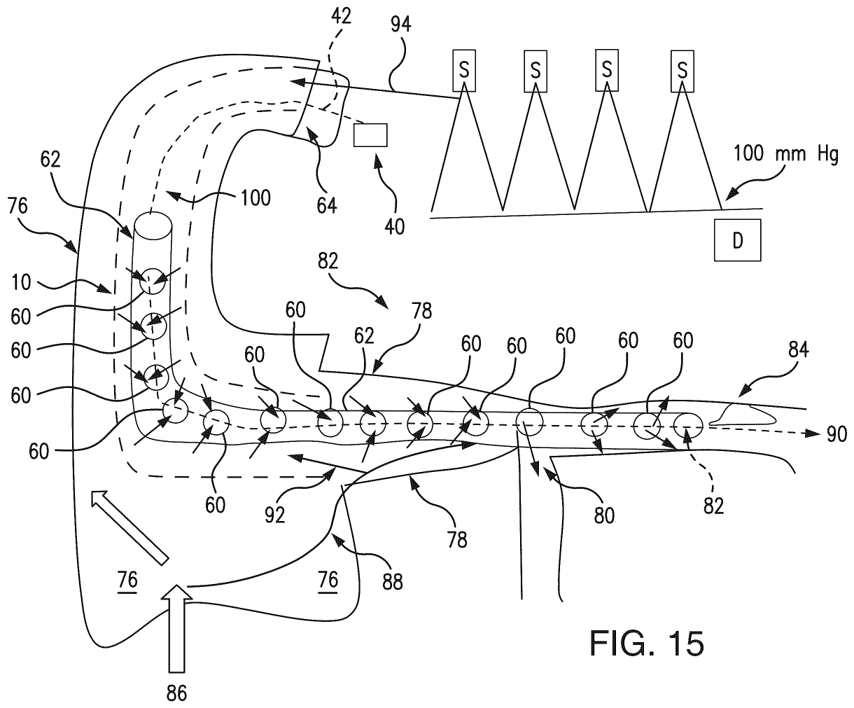

As shown in FIG. 15, during the coronary stenting, the guide catheter 10 is advanced into the aorta 76 and is engaged into the coronary artery 78. The perfusion tube 62 of the subject guide extension catheter 100 is advanced through the hemostatic valve 64 into the guide catheter 10. The perfusion tube 62 is advanced into the coronary artery 78, passed the side branch 80, and the tip of the perfusion tube 82 is positioned proximal to the stenosis 84. Since the pressure in the aorta 76 is higher than that that in the perfusion tube 62, blood flows in a direction 88 to the proximal coronary artery 78, perfusion ports 60, perfusion tube 62, and the distal coronary artery 90. This provides the access for the oxygenated blood to the distal coronary artery and the heart muscle during stenting procedure. In addition, blood flows in a direction 92 from the aorta 76 to the guide catheter 10, proximal perfusion ports 60, and perfusion tube 62. Given blood flows from the heart 86 to the ascending aorta 76 and guide catheter 10, the operator can assess blood pressure 94 at the hub of the guide catheter. Because of continuous blood perfusion from the aorta to the tip of perfusion tube 62 disposed proximal to the stenosis, it is expected that there will be no blood pressure drift from the hub of the guide catheter 94 to the tip of perfusion tube 82.

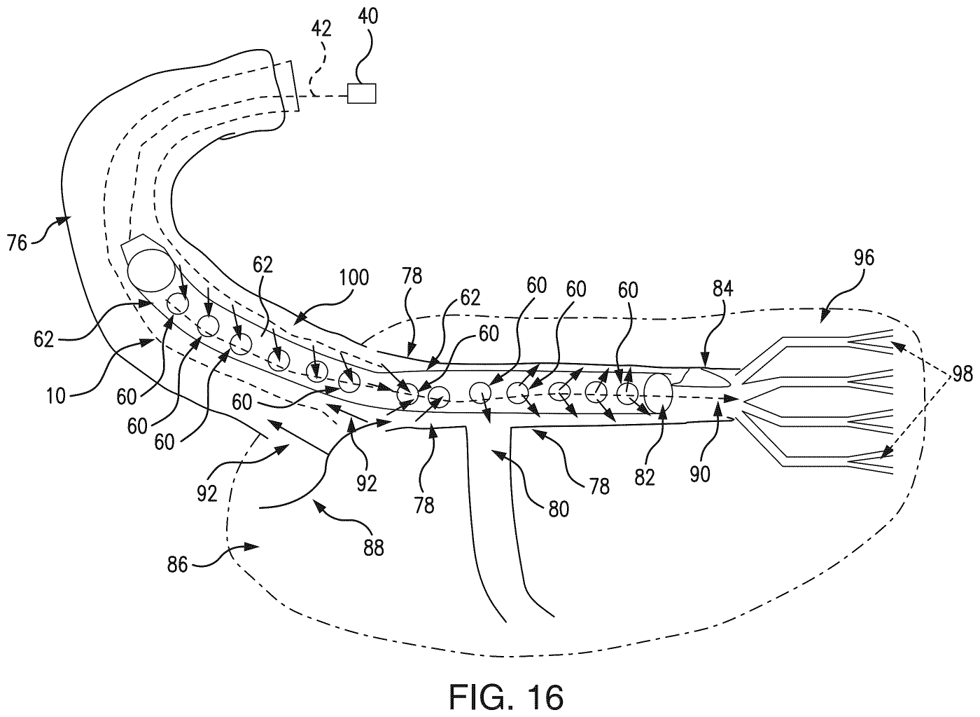

FIG. 16 shows another embodiment of the present guide extension catheter 100 displaying perfusion to the heart by the perfusion tube 62. As shown in FIG. 16, after insertion of the perfusion tube 62 into the coronary artery, blood flow to the coronary artery is expected to be in following directions: (1) Blood flows in a direction 88 from the heart 86 to the proximal coronary artery 78, perfusion ports 60, perfusion tube 62, distal coronary artery 90, coronary microvascular system (small coronary arteries) 96, and the heart tissue 98, (2) blood flows from the perfusion ports 60 to the side-branch 80 and the heart tissue around the perfusion tube, and (3) blood flows in a direction 92 from the heart 86 to the ascending aorta 76, guide catheter 10, proximal perfusion ports 60, perfusion tube 62 and distal coronary artery 90. Given that blood pressure is higher in the aorta 76 than in the perfusion tube 62, blood flows from the proximal perfusion tube 62 to its distal shaft, and then to the coronary microcirculation 96 and heart tissue 98.

Although aspects of the present disclosure have been described in connection with specific forms and embodiments thereof, it will be appreciated that various modifications other than those discussed above may be resorted to without departing from the spirit or scope of the present disclosure as defined in the appended claims. For example, functionally equivalent elements may be substituted for those specifically shown and described, certain features may be used independently of other features, and in certain cases, particular locations of the elements may be reversed or interposed, all without departing from the spirit or scope of the present disclosure as defined in the appended claims.

What is claimed is:

1. A guide extension catheter system advanceable through a guide catheter to provide back-up support for coronary stent deployment in a blood vessel, comprising:
a proximal section, a distal section, and a middle section disposed between said proximal and distal sections,
a push handle disposed at the proximal section, an alignment device disposed at the middle section, and a perfusion tube disposed at the distal section,
the perfusion tube having a cylindrically shaped shaft extending between a proximal end and a distal end thereof, wherein said proximal end of said cylindrically shaped shaft of the perfusion tube is configured with a concave-shaped inlet, and wherein said cylindrically shaped shaft has an internal lumen and a wall defining the internal lumen and configured with a plurality of perfusion ports formed in the wall of the cylindrically shaped shaft, the plurality of perfusion ports being disposed spirally around and along a length of the cylindrically shaped shaft of the perfusion tube.

2. The guide extension catheter system of claim 1, further comprising a stent system insertable longitudinally in said lumen of said cylindrically shaped shaft and displaceable therealong between the proximal section of said guide extension catheter system and beyond the distal end of the cylindrically shaped shaft of the perfusion tube, and a guide catheter configured to receive the guide extension catheter longitudinally therein for displacement therealong, wherein the stent enters the lumen of the perfusion tube through the concave—shaped inlet formed at the proximal end of the cylindrically shaped shaft of the perfusion tube.

3. The guide extension catheter of claim 2, wherein the perfusion tube has a cross-sectional outer diameter smaller than a cross-sectional outer diameter of the guide catheter.

4. The guide extension catheter of claim 1, wherein said perfusion ports formed in the wall of the cylindrically shaped shaft of the perfusion tube provide a directed fluid communication from the proximal end to and through the distal end of the perfusion tube.

5. The guide extension catheter of claim 4, wherein the perfusion tube is insertable into a coronary artery, and wherein the plurality of perfusion ports support delivery of oxygenated blood to the heart muscle.

6. The guide extension catheter of claim 2, wherein the push handle has an outer diameter smaller than an outer diameter of the perfusion tube, wherein a length of the push handle combined with the lengths of the cylindrically shaped shaft of the perfusion tube and a length of the alignment device forms a collective length of the guide extension catheter, wherein the collective length of the guide extension catheter exceeds a length of the guide catheter.

7. The guide extension catheter of claim 6, wherein the push handle is configured to transfer an action applied thereto to the alignment device and to the perfusion tube to advance the perfusion tube into the coronary artery, and wherein, when the guide extension catheter is disposed in the guide catheter, the cylindrically shaped shaft of the perfusion tube extends out of a distal end of the guide catheter, and a proximal end of the push handle extends out of the guide extension catheter at the proximal section thereof.

8. The guide extension catheter of claim 1, wherein the cylindrically shaped shaft of the perfusion tube is configured with an outer layer and inner layer formed in contact with an outer surface and an inner surface of the wall of the cylindrically shaped shaft, respectively, the cylindrically shaped shaft of the perfusion tube further comprising a reinforcement structure configured with spiral coils embedded between the outer and inner layers of the cylindrically shaped shaft of the perfusion tube to prevent kinking or collapse of the perfusion tube during advancing into the blood vessel.

9. The guide extension catheter of claim 2, wherein said cylindrically shaped shaft of the perfusion tube further includes a reinforcing polymer layer secured to the wall of the cylindrically shaped shaft at the proximal end thereof and having a length of approximately 2 cm, said reinforcing polymer layer provides strength, pushability and flexibility to the perfusion tube to support passage of the stent into the perfusion tube.

10. The guide extension catheter of claim 1, wherein the cylindrically shaped shaft of the perfusion tube is formed with a distal tip at the distal end of the cylindrically shaped shaft, the distal tip having a length of approximately 2 mm and being formed from a soft radiopaque material to provide atraumatic advancement of the perfusion tube into the blood vessel.

11. The guide extension catheter of claim 8, wherein a diameter of the lumen of the cylindrically shaped shaft of the perfusion tube is sufficient for passage of interventional devices therethrough and for supporting sufficient perfusion to the heart.

12. The guide extension catheter of claim 8, therein the outer layer has a hydrophilic coating for smooth passage of the perfusion tube into the blood vessel.

13. The guide extension catheter of claim 1, wherein the perfusion ports are formed in the wall of the cylindrically shaped shaft along entire length thereof.

14. The guide extension catheter of claim 8, wherein the perfusion ports are disposed between adjacent windings of the spiral coils of the cylindrically shaped shaft of the perfusion tube.

15. The guide extension catheter of claim 1, wherein the perfusion ports are configured in a variety of geometric shapes, including a round shape, and have a diameter ranging from 0.014 to 0.020 inches.

16. The guide extension catheter of claim 5, wherein the perfusion ports are disposed along the cylindrically shaped shaft of the perfusion tube to maintain coronary perfusion and to prevent hydraulic dissection of the coronary artery and heart attack.

17. The guide extension catheter of claim 1, wherein the alignment mechanism comprises a U-shaped polymer member and said concave-shaped inlet in direct coupling with said U-shaped polymer member.

18. The guide extension catheter of claim 17, wherein the U-shaped polymer member has a proximal end and a distal end, said distal end of the U-shaped polymer member being incorporated in said concave-shaped inlet.

19. The guide extension catheter of claim 18, further including a guidewire, wherein a top portion of the U-shaped polymer member is cutout to prevent wrapping of the guidewire and to facilitate the passage of interventional devices to the concave-shaped inlet without entanglement.

20. The guide extension catheter of claim 1, wherein the concave-shaped inlet is formed with a polymer to maintain flexibility of and to prevent collapse of the lumen of the cylindrically shaped shaft.

21. A method for intravascular treatment of a coronary artery blockage comprising:
(a) advancing a guide catheter through aorta into the ostium of the coronary artery;
(b) advancing a guidewire through a lumen of the guide catheter into the coronary artery and crossing beyond the blockage;
(c) assembling a guide extension catheter having a push handle disposed at a proximal section of the guide extension catheter, an alignment device at a middle section of the guide extension catheter, and a perfusion tube at a distal section of the guide extension catheter, the perfusion tube having a concave-shaped inlet at a proximal end thereof and a plurality of perfusion ports disposed spirally along a length of the perfusion tube;
(d) advancing the push handle to position the perfusion tube beyond a distal end of the guide catheter into the coronary artery by using the guidewire as a rail;
(e) advancing a balloon angioplasty catheter over the guidewire through the alignment mechanism, and positioning the perfusion tube proximal to the blockage in the coronary artery; and
(f) advancing a stent over the guidewire through the alignment mechanism and the perfusion tube into the coronary artery and positioning the stent across the blockage to unblock the blockage.

22. The method as recited in claim 21, further comprising:
in said step (e), advancing a guidewire and interventional devices through the alignment device into the perfusion tube with no snagging or stripping off of the stent from balloon angioplasty catheter.

23. The method as recited in claim 21, further comprising:
in said step (d), advancing the perfusion tube into the coronary artery distal to the side-branch and positioning it proximal to the stenosis.

24. The method as recited in claim 21, further comprising:
in said step (e), configuring blood flow from the heart and passing through the aorta, proximal coronary artery, perfusion ports, perfusion tube, distal coronary artery, coronary microvasculature and heart tissue.

25. The method as recited in claim 24, further comprising maintaining continuous perfusion of the distal coronary artery by the perfusion tube, thus reducing the risk of ischemia of the heart muscle.

26. The method as recited in claim 25, further comprising maintaining perfusion from a proximal end to a distal end of the perfusion tube, thus preventing blood pressure drift from a hub of the guide catheter to a distal tip of the perfusion tube.

27. The method as recited in claim 24, further comprising providing oxygenated blood to a side-branch by the perfusion ports and preventing ischemia in the side-branch.

28. The method as recited in claim 24, further comprising maintaining continuous perfusion of the distal coronary artery by the perfusion tube, thus reducing the risk of hydraulic dissection with contrast injection.
